(19) United States
(12) Patent Application Publication
(10) Pub. No.: US 2016/0033522 A1
(43) Pub. Date: Feb. 4, 2016

(54) METHYLCITRATE ANALYSIS IN DRIED BLOOD SPOTS

(71) Applicants: Osama Al-Dirbashi, Ottawa (CA); CHILDREN'S HOSPITAL OF EASTERN ONTARIO, Ottawa (CA)

(72) Inventors: Osama AL-DIRBASHI, Ottawa (CA); Pranesh CHAKRABORTY, Ottawa (CA)

(21) Appl. No.: 14/791,335

(22) Filed: Jul. 3, 2015

(30) Foreign Application Priority Data
Aug. 1, 2014 (GB) 1413703.8

(51) Int. Cl. G01N 33/64

(52) U.S. Cl. CPC G01N 33/64; G01N 2800/04; G01N 2560/00

(57) ABSTRACT

There is provided a method of measuring methylcitrate (MCA) in a sample by derivatizing the MCA, for example with 4-[2-(N,N-dimethylamino)ethylaminosulfonyl]-7-(2-aminoethylamino)-2,1,3-benzoxadiazole (DAABD-AE); measuring a level of the derivatized MCA; and determining the level of MCA from the measured level. The sample may be a dried blood spot (DBS), and the extraction and derivatization may be carried out simultaneously. No separate extraction or purification step is required, thereby reducing sample handling. Measuring may be carried out by mass spectrometry. The method may be used to screen for subjects having or at increased risk of a propionylcarnitine (C3) related disorder. The method may be used as a first tier screen, or as a second tier test for a sample that previously triggered a positive result in a primary screen. The method may be applied to newborn screening. Related kits and uses are also provided.

### FIELD

This application claims the benefit of priority of GB Patent Applicant No. 1413703.8 filed Aug. 1, 2014, which is incorporated herein by reference in its entirety.

The present disclosure relates generally to measuring metabolites. More particularly, the present disclosure relates to measuring methylcitrate.

### BACKGROUND

Accumulation of propionate metabolites in body fluids can be caused by defects in propionyl coenzyme A or cobalamin (Cbl) metabolism (Fenton et al. 2001). These metabolites are chiefly derived from the catabolism of certain amino acids and odd chain fatty acids. Propionate accumulation results in increased propionylcarnitine (C3) concentration in biological samples. C3 is a marker of a number of inborn errors of metabolism, including methylmalonyl CoA mutase deficiency (OMIM ID: 251000) and propionic acidemia (PA, OMIM ID: 606054) (Zytkovicz et al. 2001; Rashed et al. 1997). Elevated C3 can also be associated with defects in several other genes involved in Cbl metabolism (Fowler et al. 2008; Watkins et al. 2011). A subset of C3 related metabolic disorders has been recommended as screening targets and are widely adopted by newborn screening programs around the world (Watson et al. 2006). Clinically, these are often accompanied by episodic metabolic acidosis, ketosis, hyperammonemia, and may result in severe sequelae including neurological symptoms or death in infancy. Early diagnosis is key to effective management and favorable outcome is expected should treatment start before the appearance of symptoms (Hori et al. 2005; Schulze et al. 2009).

In neonatal dried blood spot (DBS) specimens used in screening, the concentration of C3 in affected newborns overlaps with healthy individuals rendering screening for relevant disorders using this metabolic intermediate as a sole marker neither specific nor sensitive. In Ontario, screening for PA and MMA started in 2006 based on C3 and one baby with a false negative screen (methylmalonyl-CoA mutase deficiency) was identified among 507,428 infants screened in the first 4 years. By adjusting C3 cutoff and introducing the ratio of C3 to acetylcarnitine (C3/C2), the sensitivity and specificity was improved (Wilcken et al. 2003; Chace et al. 2003; Lindner et al. 2008). However, the positive predictive value remained generally poor (La Marca et al. 2007).

In some approaches, screen positive newborns are recalled for further diagnostic workup. Simultaneous testing of mothers to eliminate a maternal condition is also not uncommon and most of these babies turn out to be unaffected. The high false positive rate negatively impacts the cost-benefit ratio of newborn screening, leads to parental anxiety and increases risk for parent-child dysfunction (Gurian et al. 2006). Unfortunately, routine MS/MS-based newborn screening methodology is inadequate to reduce the false positive rate and new analytical strategies are needed to improve the screening performance.

In an article entitled “Validated capillary gas chromatographic-mass spectrometric assay to determine 2-methylcitrate acid I and II levels in human serum (Journal of Chromatography B (2000), vol. 775: 215-223), Busch et al. disclose an assay for 2-methylcitric acid. Human serum is the starting point for the assay. Samples are first fractionated using anionic exchange chromatography, vacuum dried for 90 minutes at 40° C., and derivatized by incubating for 40 minutes at 90° C. with N-methyl-N(tert.-butyldimethyl-silyl)-trifluoroacetamide (MBDSTFA). Samples are then analyzed by gas chromatography/mass spectrometry (GC-MS).

WO93/01496 A1 to the University of Colorado Foundation Inc. discloses, on pages 17 to 19, an assay for 2-methylcitrate. The method employs 400 μL serum, 400 μL cerebral spinal fluid, or 40 μL of urine. Samples are extracted, and then fractionated by anion exchange chromatography. Eluates are dried by vacuum centrifugation. The dried eluates are derivatized by adding MBDSTFA and incubating at 90° C. for 30 minutes. Samples analysis is carried out by GC-MS.

In an article entitled “Methylcitric acid determination in amniotic fluid by electron-impact mass fragmentography” (Journal of Clinical Chemistry and Clinical Biochemistry (1988), vol. 26: 345-348), Kretschmer et al. describe a method of measuring methylcitric acid in amniotic fluid. Samples were absorbed on an Extrelut-3 column for 10 minutes, then eluted into a flask. Samples were then dried, prior to addition of N-(t-butyldimethylsilyl)trifluoroacetamide and incubation at 70° C. for one hour. Samples were then injected into a GC-MS system.

In an article entitled “Determination of Total Homocysteine, Methylmalonic Acid, and 2-Methylcitric Acid in Dried Blood Spots by Tandem Mass Spectrometry” (Clinical Chemistry (2010), vol. 56(11): 1686-1695), Turgeon et al. describe a method for measuring MCA in a dried blood spot. A 4.8 mm diameter disc was punched from a dried blood spot. Sample preparation included addition of a solvent, extraction (60 minutes), transferal of the eluate to a new vial, drying (15 to 20 minutes), derivatization with n-butanol HCl (15 minutes), and evaporation of excess reagent (5 to 7 minutes) prior to analysis by liquid chromatography—tandem mass spectrometry (LC-MS/MS).

It is desirable to screen for C3 related disorders.

### SUMMARY

It is an object of the present disclosure to obviate or mitigate at least one disadvantage of previous approaches.

In a first aspect, the present disclosure provides a method of measuring a level of 2-methylcitric acid (MCA) in a sample, comprising derivatizing the MCA; measuring the derivatized MCA; and determining the level of MCA from the level of derivatized MCA.

In another aspect, there is provided a method of measuring a level of 2-methylcitric acid (MCA) in a sample, comprising derivatizing the MCA with 4-[2-(N,N-dimethylamino)ethylaminosulfonyl]-7-(2-aminoethylamino)-2,1,3-benzoxadiazole (DAABD-AE); measuring the derivatized MCA; and determining the level of MCA from the level of derivatized MCA.

In another aspect, there is provided a method of screening for a subject having an increased risk of a propionylcarnitine (C3) related disorder comprising derivatizing 2-methylcitric acid (MCA) of sample obtained from a subject; measuring a level of the derivatized MCA; comparing the level to a threshold, and determining that the subject has an increased risk of having a C3 related disorder in response to a determination that the level exceeds the threshold.

In another aspect, there is provided a method of screening for a subject having an increased risk of a propionylcarnitine (C3) related disorder comprising derivatizing 2-methylcitric acid (MCA) of sample obtained from a subject with DAABD-AE; measuring a level of the derivatized MCA; comparing the level to a threshold, and determining that the subject has an increased risk of having a C3 related disorder in response to a determination that the level exceeds the threshold.

In another aspect, there is provided a kit for use in measuring a level of 2-methylcitric acid (MCA) in a sample, comprising: a derivatizing agent; and instructions for derivatizing MCA with the derivatizing agent, measuring a level of the derivatized MCA; and determining the level of MCA from the measured level of derivatized MCA.

In another aspect, there is provided a kit for use in measuring a level of 2-methylcitric acid (MCA) in a sample, comprising: DAABD-AE; and instructions for derivatizing MCA with the DAABD-AE, measuring a level of the derivatized MCA; and determining the level of MCA from the measured level of derivatized MCA.

In another aspect, there is provided a diagnostic kit for use in screening for a subject having an increased risk of a C3 related disorder comprising: a derivatizing agent; and instructions for: derivatizing the MCA with the derivatizing agent, measuring a level of the derivatized MCA, comparing the level to a threshold, and determining that the subject has an increased risk of having a C3 related disorder in response to a determination that the level exceeds the threshold.

In another aspect, there is provided a diagnostic kit for use in screening for a subject having an increased risk of a C3 related disorder comprising: DAABD-AE; and instructions for: derivatizing the MCA with the DAABD-AE, measuring a level of the derivatized MCA, comparing the level to a threshold, and determining that the subject has an increased risk of having a C3 related disorder in response to a determination that the level exceeds the threshold.

In one aspect, there is provided a use of a derivatizing agent for preparation of derivatized MCA from a sample from a subject for determining a level of MCA.

In one aspect, there is provided a use of DAABD-AE for preparation of derivatized MCA from a sample from a subject for determining a level of MCA.

In one aspect, there is provided a use a derivatizing agent for preparation of derivatized MCA from a sample from a subject for determining a level of MCA.

In one aspect, there is provided a use of DAABD-AE for preparation of derivatized MCA from a sample from a subject for determining a level of MCA.

In another aspect, there is provided a use of derivatized MCA obtained from a sample from a subject for determining a level of MCA.

In another aspect, there is provided a use of DAABD-AE derivatized MCA obtained from a sample from a subject for determining a level of MCA.

In another aspect, there is provided a use of a derivatizing agent for preparation of derivatized MCA from a sample from a subject for determining a risk of a propionylcarnitine (C3) related disorder of the subject.

In another aspect, there is provided a use of DAABD-AE for preparation of derivatized MCA from a sample from a subject for determining a risk of a propionylcarnitine (C3) related disorder of the subject.

In another aspect, there is provided a use of derivatized MCA obtained from a sample from a subject for determining a risk of a C3 related disorder of the subject.

In another aspect, there is provided a use of DAABD-AE derivatized MCA obtained from a sample from a subject for determining a risk of a C3 related disorder of the subject.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts ESI-MS spectra. Panel A depicts ESI-MS spectrum of DAABD-MCA derivative, while Panel B depicts an ESI-MS/MS spectrum of m/z 499.

FIG. 2 depicts extracted mass chromatograms. Panel A shows a chromatogram obtained with a DBS from a healthy individual, while Panel B shows a chromatogram obtained from a DBS from a methylmalonic aciduria (MMA) patient. Solid lines represent MCA and dotted lines represent the IS. The arrow points at MCA in the healthy individual's sample.

FIG. 3 shows the stability of MCA in DBS samples stored at different temperatures.

FIG. 4 shows a comparison of MCA concentrations in DBS (n=252) obtained using a described herein versus a reference method after modification (Turgeon et al. 2010).

### DETAILED DESCRIPTION

Generally, the present disclosure provides a screen for C3 related disorders based on measuring 2-methylcitric acid (MCA), a pathognomonic hallmark of C3 related disorders.

Methods

In a first aspect, the present disclosure provides a method of measuring a level of 2-methylcitric acid (MCA) in a sample, comprising derivatizing the MCA; measuring the derivatized MCA; and determining the level of MCA from the level of derivatized MCA.

In one aspect, the present disclosure provides a method of measuring a level of 2-methylcitric acid (MCA) in a sample, comprising derivatizing the MCA with 4-[2-(N,N-dimethylamino)ethylaminosulfonyl]-7-(2-aminoethylamino)-2,1,3-benzoxadiazole (DAABD-AE); measuring the derivatized MCA; and determining the level of MCA from the level of derivatized MCA.

In one embodiment, the sample is from a dried blood spot (DBS). For example, the sample may be a punch from a dried blood spot. The punch may have a size of less than 18 mm². For example, the punch may be less than 17 mm², less than 16 mm², less than 15 mm², less than 14 mm², less than 13 mm², less than 12 mm², less than 11 mm², less than 10 mm², less than 9 mm², less than 8 mm², less than 7 mm², less than 6 mm², or less than 5 mm². In one particular embodiment, the punch is less than 10 mm². In another embodiment, the punch is about 7 mm².

In one embodiment, the step of measuring is carried out by mass spectrometry, which may be liquid chromatography tandem mass spectrometry (LC-MS/MS).

The poor ionization and fragmentation of MCA in electrospray ionization (ESI)-MS/MS can be improved by derivatization prior to mass spectrometry, in some embodiments. For example, MCA may be derivatized with 4-[2-(N,N-dimethylamino)ethylaminosulfonyl]-7-(2-aminoethylamino)-2,1,3-benzoxadiazole (DAABD-AE). DAABD-AE is particularly advantageous because of its high reactivity. This eliminates the need for sample extraction required with other methods, e.g. with butanolic-HCl. Derivatization with DAABD-AE also allows the screen to be completed in one day in some embodiments. In some embodiments, the sensitivity obtained with DAABD-AE is superior.

In one embodiment, the MCA may be derivatized with DBAAD-AE for less than an hour. For example, MCA may be derivatized with DBAAD-AE for less than 60 minutes, less than 50 minutes, less than 40 minutes, less than 30 minutes, less than 20 minutes, or less than 10 minutes. In one particular embodiment, MCA is derivatized with DAABD-AE for about 45 minutes. In some embodiments, the method may be completed within the course of one day, or half a day. The method may be applied to multiple samples, e.g. in parallel, and may be completed in one day, or half a day.

In one embodiment, no separate extraction or purification step is required prior to the step of measuring. Extraction and derivatization may be performed simultaneously in some embodiments. In some embodiments, the derivatized/extracted sample may be used directly for mass spectrometry analysis.

In one embodiment, derivatization and extraction may be performed directly using a 3.2-mm disc of DBS as a sample (e.g, at 65° C. for 45 min).

Other reagents that may be used to derivatize MCA in some embodiments include, for example: HCl-butanol, trimethylamino-ethylalcohol (TAME), or 4-dimethylamino-benzylamine.

In some embodiments, the method meets Clinical Laboratory Improvement Amendments (CLIA) certification standards.

In one aspect, there is provided a method of screening for a subject having an increased risk of a propionylcarnitine (C3) related disorder comprising derivatizing 2-methylcitric acid (MCA) of a sample obtained from a subject measuring a level of the derivatized MCA; comparing the level to a threshold, and determining that the subject has an increased risk of having a propionylcarnitine (C3) related disorder in response to a determination that the level exceeds the threshold.

In one aspect, there is provided a method of screening for a subject having an increased risk of a propionylcarnitine (C3) related disorder comprising derivatizing 2-methylcitric acid (MCA) of sample obtained from a subject with 4-[2-(N,N-dimethylamino)ethylaminosulfonyl]-7-(2-aminoethylamino)-2,1,3-benzoxadiazole (DAABD-AE); measuring a level of the derivatized MCA; comparing the level to a threshold, and determining that the subject has an increased risk of having a propionylcarnitine (C3) related disorder in response to a determination that the level exceeds the threshold.

By “C3 related disorder” is meant a defect of propionyl coenzyme A metabolism, involving an abnormal amount of propionylcarnitine (C3), or a related metabolite, e.g. an increase or decrease compared to a healthy individual. For example, these may involve an increased level of C3 itself. A C3 related disorder may be a metabolic disorder. Examples include propionic acidemia (PA), a defect of cobolamin (Cbl) metabolism, and/or methylmalonic aciduria (MMA) or acidemia, such as that caused by methylmalonyl CoA mutase deficiency. Other examples also include CbIA, CbIB, CbIC, CbID, CbIF, unclassified cobalamin defects, and/or transcobalamin deficiency.

By “increased risk” is meant that the subject has a risk that is at least greater than the general population or a control population. However, the “increased risk” parameter may be adjusted to suit screening requirements. Subjects determined to have “increased risk” can be assessed by other methods to confirm the presence or absence of disease.

By “threshold” is meant a value selected to discriminate between subjects having normal risk, and subjects having increased risk. Alternatively, a “threshold” may be selected to discriminate between a disease state and a non-disease state. The threshold may be selected according to requirements, e.g. to identify subjects having a particular increased risk or e.g. to achieve a specific sensitivity, specificity, and/or positive predictive value (PPV) parameter. In some embodiments, the method has improved specificity over existing methods while maintaining excellent sensitivity.

In one embodiment, the method may be used to screen for a subject having a C3 related disorder.

2-Methylcitric acid (MCA) is generally described as a pathognomonic hallmark for disorders involving propionyl CoA metabolism. MCA can be detected in DBS and has been shown to reduce the false positive rate and improve the positive predictive value for PA and MMA (Turgeon et al. 2010). Accordingly, in some embodiments the C3 related disorder is one in which MCA is elevated.

In one embodiment, the sample is a dried blood spot (DBS). For example, the sample may be a punch from a dried blood spot. The punch may have a size of less than 18 mm². For example, the punch may be less than 17 mm², less than 16 mm², less than 15 mm², less than 14 mm², less than 13 mm², less than 12 mm², less than 11 mm², less than 10 mm², less than 9 mm², less than 8 mm², less than 7 mm², less than 6 mm², or less than 5 mm². In one particular embodiment, the punch is less than 10 mm². In another embodiment, the punch is about 7 mm².

In one embodiment, the step of measuring is carried out by mass spectrometry, which may be liquid chromatography tandem mass spectrometry (LC-MS/MS). The poor ionization and fragmentation of MCA in electrospray ionization (ESI)-MS/MS can be improved by derivatization prior to mass spectrometry.

MCA may be derivatized with 4-[2-(N,N-dimethylamino)ethylaminosulfonyl]-7-(2-aminoethylamino)-2,1,3-benzoxadiazole (DAABD-AE). DAABD-AE is particularly advantageous because of its high reactivity, which eliminates the need for sample extraction required, e.g. with butanolic-HCl. Derivatization with DAABD-AE also allows the screen to be completed in one day. In some embodiments, the sensitivity obtained with DAABD-AE is superior.

In one embodiment, the MCA may be derivatized with DBAAD-AE for less than an hour. For example, MCA may be derivatized with DBAAD-AE for less than 60 minutes, less than 50 minutes, less than 40 minutes, less than 30 minutes, less than 20 minutes, or less than 10 minutes. In one particular embodiment, MCA is derivatized with DAABD-AE for about 45 minutes. In some embodiments, the method may be completed within the course of one day, or half a day. The method may be applied to multiple samples, e.g. in parallel, and may be completed in one day, or half a day.

In one embodiment, no separate extraction step is required prior to the step of measuring. In one embodiment, no purification step is required prior to the step of measuring. Extraction and derivatization may be performed simultaneously in some embodiments. In some embodiments, the derivatized/extracted sample may be used directly for mass spectrometry analysis.

In one embodiment, derivatization and extraction may be performed directly using a 3.2-mm disc of DBS as a sample (e.g., at 65° C. for 45 min).

In one particular embodiment, the reaction mixture was analyzed by liquid chromatography tandem mass spectrometry. MCA was well separated and eluted at 2.3 min with a total run time of 7 min.

Other reagents that may be used to derivatize MCA in some embodiments include, for example: HCl-butanol, trimethylamino-ethylalcohol (TAME), or 4-dimethylamino-benzylamine.

In one embodiment, the subject may be a newborn infant. The subject may be one that previously screened positive for a C3 related disorder in a primary screen. The method may be applied as a second tier test for samples which trigger positive results, e.g. PA or MMA, results by a primary newborn screening method. Such second tier testing, whereby the initial screening sample is tested for a different marker or using alternative technology, can be efficient in improving specificity (Minutti et al. 2004; Janzen et al. 2007).

The superior sensitivity attained in some embodiments allows for MCA detection, e.g., using a single 3.2 mm disc. In a newborn screening laboratory setting, this method can be used for quantifying MCA in the original screening DBS to provide additional analytical information and reduce the number of false positive results.

As mentioned, a threshold may be set according to requirements. In some embodiments, the threshold cutoff for MCA is 0.1 μmol/L, 0.2 μmol/L, 0.3 μmol/L, 0.4 μmol/L, 0.5 μmol/L, 0.6 μmol/L, 0.7 μmol/L, 0.8 μmol/L, 0.9 μmol/L, 1.0 μmol/L, 1.1 μmol/L, 1.2 μmol/L, 1.3 μmol/L, 1.4 μmol/L, 1.5 μmol/L, 1.6 μmol/L, 1.7 μmol/L, 1.8 μmol/L, 1.9 μmol/L, or 2.0 μmol/L. In one particular embodiment the threshold cutoff for MCA is 0.8 μmol/L. In one particular embodiment the threshold cutoff for MCA is 1.0 μmol/L. For instance, a lower threshold (such as 0.8 μmol/L) may be used when detection of transcobalamin II deficiency is desirable, while a higher threshold (such as 1.0 μmol/L) may be used when its detection is not a concern, or when it is desirable to attain a higher positive predictive value.

In one embodiment, the method may further comprise measuring other metabolites associated with C3 related disorders. For instance, the method may further comprise measuring propionylcarnitine (C3) and measuring acetylcarnitine (C2). In some embodiment, the method may further comprise determining a ratio of C3/C2.

In one embodiment, the method may be used to identify a C3 related disorder in a subject.

In one embodiment, the subject is a newborn infant.

In one embodiment, the method may be used to screen for a C3 related disorder, for example, as defined above. The C3 related disorder may be a defect of propionyl coenzyme A metabolism. For example, the C3 related disorder may be propionic acidemia (PA), a defect of cobolamin (Cbl) metabolism, or methylmalonic aciduria (MMA) or acidemia, such as that caused by methylmalonyl CoA mutase deficiency. Other examples, in some embodiments, include CbIA, CbIB, CbIC, CbID, CbIF, unclassified cobalamin defects, and/or transcobalamin deficiency.

In some embodiments, addition of the above-described method to a screening method based on C3 and or C3/C2 ratio may improve the positive predictive value compared to screening based on C3 and/or C3/C2 ratio only. In some embodiments, the addition may reduce the number of samples that screen positive due to maternal vitamin B12 deficiency.

In one embodiment, the method further comprises measuring a level of derivatized MCA in control material. By “control material” is meant any material that contains a known or pre-determined amount of MCA. Such material may include, for example, calibration samples with known amounts of MCA that permit a standard curve to be established. The control material may also comprise a quality control sample, e.g. to permit comparison of results obtained with different batches or production lots.

In some embodiments, the above-described methods meet Clinical Laboratory Improvement Amendments (CLIA) certification standards. In some embodiments, the above-described methods are useful in high volume screening. For examples, the above-described methods may have reduced processing complexity (e.g. fewer preparatory steps), may require less sample handling (e.g. less transferring between sample tubes), may require less time to perform (e.g. may be completed within a day), require less starting material (e.g. leaving material from dried blood spot available for other tests), and/or are amenable to starting material routinely available for newborn screening (e.g. a dried blood spot).

In some embodiments, the above-described methods exhibit improved sensitivity and/or improve the sensitivity of existing methodologies. For example, when paired with testing for C3 and C2, the above-described methods may have a positive prediction value of greater than 11%, 15%, 20%, 25%, or 30%. In some embodiments, when coupled with testing for C3 and C2, the above described methods may yield a positive prediction value of about 33%. In these embodiments, the above-described methods have the potential to reduce unnecessary patient referrals, e.g. by about three-fold.

In some embodiments, the above-described methods could be used to monitor the efficacy of treatment or therapy. For instance, a reduction in MCA may provide an indication of efficacy of treatment or therapy.

Kits

In another aspect, there is provided a kit for carrying out the above-described methods.

In one aspect, there is provided a kit for use in measuring a level of 2-methylcitric acid (MCA) in a sample, comprising: a derivatizing agent; and instructions for derivatizing MCA with the DAABD-AE, measuring a level of the derivatized MCA; and determining the level of MCA from the measured level of derivatized MCA.

In one aspect, there is provided a kit for use in measuring a level of 2-methylcitric acid (MCA) in a sample, comprising: 4-[2-(N,N-dimethylamino)ethylaminosulfonyl]-7-(2-aminoethylamino)-2,1,3-benzoxadiazole (DAABD-AE); and instructions for derivatizing MCA with the DAABD-AE, measuring a level of the derivatized MCA; and determining the level of MCA from the measured level of derivatized MCA.

In one embodiment, the sample may be from a dried blood spot (DBS). In one embodiment, the sample may be a punch from a dried blood spot. The punch may have a size of less than 18 mm². The punch may have a size of less than 10 mm². The punch may have a size of about 7 mm².

In one embodiment, the measuring may be carried out by mass spectrometry. For instance, the mass spectrometry may be liquid chromatography tandem mass spectrometry (LC-MS/MS).

In one embodiment, the instructions may indicate that the MCA is to be derivatized with the DAABD-AE for less than an hour. For instance, the instructions may indicate that the MCA is to be derivatized with the DAABD-AE for about 45 minutes.

In one embodiment, the instructions indicate that no separate extraction step is to be carried out prior to the measuring. In one embodiment, the instructions indicate that no purification step is to be carried out prior to the measuring.

In another aspect, there is provided a diagnostic kit for use in screening for a subject having an increased risk of a propionylcarnitine (C3) related disorder comprising: a derivatizing agent; and instructions for: derivatizing the MCA with the DAABD-AE, measuring a level of the derivatized MCA, comparing the level to a threshold, and determining that the subject has an increased risk of having a propionylcarnitine (C3) related disorder in response to a determination that the level exceeds the threshold.

In another aspect, there is provided a diagnostic kit for use in screening for a subject having an increased risk of a propionylcarnitine (C3) related disorder comprising: 4-[2-(N,N-dimethylamino)ethylaminosulfonyl]-7-(2-aminoethylamino)-2,1,3-benzoxadiazole (DAABD-AE); and instructions for: derivatizing the MCA with the DAABD-AE, measuring a level of the derivatized MCA, comparing the level to a threshold, and determining that the subject has an increased risk of having a propionylcarnitine (C3) related disorder in response to a determination that the level exceeds the threshold.

In one embodiment, the diagnostic kit is for use in screening for a subject having a C3 related disorder and the step of determining comprises determining that the subject has a C3 related disorder in response to a determination that the level exceeds the threshold.

In one embodiment, the sample is from a dried blood spot (DBS). In one embodiment, the sample is a punch from a dried blood spot. The punch may have a size of less than 18 mm². The punch may have a size of less than 10 mm². The punch may have a size of about 7 mm².

In one embodiment, the measuring is carried out by mass spectrometry. For instance, the mass spectrometry may be liquid chromatography tandem mass spectrometry (LC-MS/MS).

In one embodiment, the instructions indicate that the MCA is to be derivatized with the DAABD-AE for less than an hour. For instance, the instructions may indicate that the MCA is to be derivatized with DAABD-AE for about 45 minutes.

In one embodiment, the instructions indicate that no separate extraction step is to be carried out prior to the measuring. In one embodiment, the instructions indicate that no purification step is to be carried out prior to the measuring. In some embodiments, the derivatized/extracted sample may be used directly for mass spectrometry analysis.

In one embodiment, the subject is a newborn infant.

In some embodiments, the threshold cutoff for MCA is 0.1 μmol/L, 0.2 μmol/L, 0.3 μmol/L, 0.4 μmol/L, 0.5 μmol/L, 0.6 μmol/L, 0.7 μmol/L, 0.8 μmol/L, 0.9 μmol/L, 1.0 μmol/L, 1.1 μmol/L, 1.2 μmol/L, 1.3 μmol/L, 1.4 μmol/L, 1.5 μmol/L, 1.6 μmol/L, 1.7 μmol/L, 1.8 μmol/L, 1.9 μmol/L, or 2.0 μmol/L. In one particular embodiment the threshold cutoff for MCA is 0.8 μmol/L. In one particular embodiment the threshold cutoff for MCA is 1.0 μmol/L. For instance, a lower threshold (such as 0.8 μmol/L) may be used when detection of transcobalamin II deficiency is desirable, while a higher threshold (such as 1.0 μmol/L) may be used when its detection is not a concern, or when it is desirable to attain a higher positive predictive value.

In one embodiment, the diagnostic kit is for use in a screening assay that comprises measuring propionylcarnitine (C3) and measuring acetylcarnitine (C2).

In one embodiment, the subject previously screened positive for a C3 related disorder in a primary screen.

In one embodiment, the C3 related disorder is a defect of propionyl coenzyme A metabolism. For example, the C3 related disorder may be propionic acidemia (PA), a defect of cobolamin (Cbl) metabolism, or methylmalonic aciduria (MMA) or acidemia, such as that caused by methylmalonyl CoA mutase deficiency. Other examples, in some embodiments, include CbIA, CbIB, CbIC, CbID, CbIF, unclassified cobalamin defects, and/or transcobalamin deficiency.

In one embodiment, the diagnostic kit further comprises control material. For example, the control material may comprise calibration samples for establishing a standard curve. The control material may comprise a quality control sample.

In a further embodiment, there is provided a kit for use in carrying out the above-described methods.

Other reagents that may be used as derivatizing agents for MCA in some embodiments include, for example: HCl-butanol, trimethylamino-ethylalcohol (TAME), or 4-dimethylamino-benzylamine.

Uses

In another aspect, there is provided use relating to the above methods.

In one aspect, there is provided a use of a derivatizing agent for preparation of derivatized MCA from a sample from a subject for determining a level of MCA.

In one aspect, there is provided a use of 4-[2-(N,N-dimethylamino)ethylaminosulfonyl]-7-(2-aminoethylamino)-2,1,3-benzoxadiazole (DAABD-AE) for preparation of derivatized MCA from a sample from a subject for determining a level of MCA.

In another aspect, there is provided a use of derivatized MCA obtained from a sample from a subject for determining a level of MCA.

In another aspect, there is provided a use of 4-[2-(N,N-dimethylamino)ethylaminosulfonyl]-7-(2-aminoethylamino)-2,1,3-benzoxadiazole (DAABD-AE) derivatized MCA obtained from a sample from a subject for determining a level of MCA.

In another aspect, there is provided a use of a derivatizing agent for preparation of derivatized MCA from a sample from a subject for determining a risk of a propionylcarnitine (C3) related disorder of the subject.

In another aspect, there is provided a use of 4-[2-(N,N-dimethylamino)ethylaminosulfonyl]-7-(2-aminoethylamino)-2,1,3-benzoxadiazole (DAABD-AE) for preparation of derivatized MCA from a sample from a subject for determining a risk of a propionylcarnitine (C3) related disorder of the subject.

In another aspect, there is provided a use of derivatized MCA obtained from a sample from a subject for determining a risk of a propionylcarnitine (C3) related disorder of the subject.

In another aspect, there is provided a use of 4-[2-(N,N-dimethylamino)ethylaminosulfonyl]-7-(2-aminoethylamino)-2,1,3-benzoxadiazole (DAABD-AE) derivatized MCA obtained from a sample from a subject for determining a risk of a propionylcarnitine (C3) related disorder of the subject.

In one embodiment of the above uses, the sample is from a dried blood spot (DBS). For instance, the sample may be a punch from a dried blood spot. The punch may have a size of less than 18 mm². The punch may have a size of less than 10 mm². The punch may have a size of about 7 mm².

In one embodiment of the above uses, the C3 related disorder is a defect of propionyl coenzyme A metabolism. For example, the C3 related disorder may be propionic acidemia (PA), a defect of cobolamin (Cbl) metabolism, or methylmalonic aciduria (MMA) or acidemia, such as that caused by methylmalonyl CoA mutase deficiency. Other examples, in some embodiments, include CbIA, CbIB, CbIC, CbID, CbIF, unclassified cobalamin defects, and/or transcobalamin deficiency.

Other reagents that may be used as derivatizing agents for MCA in some embodiments include, for example: HCl-butanol, trimethylamino-ethylalcohol (TAME), or 4-dimethylamino-benzylamine.

### Example 1

### Materials and Methods

Chemicals and Standard Solutions

MCA and d3-MCA used as internal standard (IS) were obtained from CDN Isotopes (Pointe-Claire, QC, Canada). DAABD-AE was synthesized according to the published method (Tsukamoto et al. 2005) but can also be purchased from Sigma-Aldrich (St. Louis, Mo., USA). 1-Ethyl-3-(3-dimethylaminopropyl) carbodiimide (EDC), 4-(dimethylamino)pyridine (DMAP) and perfluorooctanoic acid (PFOA) were obtained from Sigma-Aldrich. HPLC grade acetonitrile was from Burdick's and Jackson (Muskegon, Mich., USA). Water used throughout this study was generated by a Milli-QUV Plus Ultra pure water system (Millipore SA, Molsheim, France).

Stock solutions of MCA and IS were prepared by dissolving the proper amounts in 50% methanol to give a concentration of 10 mmol/L. These solutions were stable for a minimum of 6 months when stored at −20° C. A working IS solution at 2.5 μmol/L was prepared in 50% acetonitrile.

Controls and Patients Samples

The Institutional Research Ethics Board of the Children's Hospital of Eastern Ontario (CHEO) granted approval to this study. DBS specimens received at Newborn Screening Ontario laboratory and producing normal profiles for all screened conditions were used to determine MCA reference range (n=337). In general, samples were collected at 24-72 hours on Whatman 903™ Specimen Collection Paper. Archived DBS samples from confirmed patients with PA (n=2), MMA (n=8), Cbl C (n=1), Cbl F deficiency (n=1) and maternal vitamin B12 deficiency (n=8) were also analyzed. These samples were stored at −80° C. from the time of the initial positive screening result.

Sample Preparation

A single 3.2 mm disc was punched from the DBS into a 2.0 mL polypropylene tube. After adding 20 μL of IS working solution, the following were added successively: 25 μL of EDC (25 mmol/L in water), 25 μL of DMAP (25 mmol/L in acetonitrile) and 50 μL of DAABD-AE (2 mmol/L in acetonitrile). The tubes were tightly capped and heated at 65° C. for 45 min. The reaction was stopped by adding 120 μL of 10% methanol containing PFOA at a concentration of 0.5 g/L. The sample tubes were centrifuged at 13000 rpm for 1 min and a 10 μL portion of the resultant supernatant was injected onto the LC-MS/MS. The derivative stability was evaluated by analyzing the reaction mixture stored at 8° C. in a tightly sealed vial at 0, 2, 4, 8, 24 and 48 h after reaction.

LC-MS/MS System

The LC-MS/MS consisted of a Waters ACQUITY Ultra Performance LC system (Waters, Milford, Mass., USA) for solvent delivery and sample introduction interfaced with a Xevo XE tandem mass spectrometer (Micromass, Manchester, UK). The ESI source was operated in the positive ion mode at a capillary voltage of 3.5 kV. Cone voltage and collision energy were 35 V and 22 eV, respectively, using argon as collision gas. The ion source and desolvation temperatures were maintained at 120 and 350° C., respectively. MCA and IS were detected by selected reaction monitoring (SRM) using transitions of mass to charge (m/z) of 499 to 151 and 502 to 151, respectively, with a dwell time of 0.07 second. Separation was performed on an ACQUITY UPLC BEH C₈ column (2.1×50 mm, 1.7 μm, Waters). Mobile phase A was 10% methanol and mobile phase B was 90% methanol, and both contained 0.5 g/L PFOA. The following gradient program was used: 0-1.3 min 98% of A, 1.3-2.6 min from 98% to 50% of A, and 2.6-2.7 min 50% A at a flow rate of 0.4 mL/min. The column was re-equilibrated with 98% of mobile phase A for 4.3 min at a flow rate of 0.65 mL/min. The injection-to-injection time was 7 min.

Method Validation

To determine the linear range, DBS calibrators were prepared by adding MCA stock solution to whole blood from healthy volunteers to yield 0.5, 1, 2, 4, 8 and 16 μmol/L. Non-enriched blood was used as a blank. Quality control (QC) samples at 2.5 and 10 μmol/L were also prepared. Calibrators and QCs were applied manually onto Whatman 903™ Specimen Collection Paper and allowed to dry at ambient temperature overnight. DBS calibrators were stored at −20° C. in sealed plastic bags with a desiccant.

The limit of quantification of MCA was calculated using DBS samples prepared from spiked blood that was serially diluted to give a final concentration of 0.1 μmol/L.

Within-day (n=19) and between-day (n=14) variations were evaluated by repeatedly analyzing QC samples. Coefficient of variation (CV %) was calculated according to the following equation [CV %=100×standard deviation/mean]. Analytical recovery was calculated using data obtained from QC samples as follow [Recovery %=100×(concentration measured−concentration in non-enriched sample)/concentration added].

Stability of MCA in DBS was assessed by storing samples spiked at 1.5 and 3.5 μmol/L at various temperatures (ambient, −20° C. and 32° C.). Analysis was carried out as described over a period of 3 weeks.

For method comparison, samples with normal and abnormal MCA levels (n=252) were analyzed in parallel by the current methods and a published method after minor modification (Turgeon et al. 2010). The modification involved extending the derivatization reaction time from 15 min to 4 hours.

### Example 2

### Sample Preparation

Extraction and derivatization of MCA was accomplished in a single step. IS and reagents required for derivatization were added directly onto a 3.2 mm DBS disc and incubated at 65° C. The derivatization yield which represents the extraction of MCA from DBS and the formation of DAABD-MCA derivative reached its maximum at 45 min or more. All following experiments hence were performed at 65° C. for 45 min. DAABD-MCA derivatives were stable for at least 48 h when stored in a tightly sealed vial at 8° C.

### Example 3

### MS/MS and LC-MS/MS Experiments

The reaction mixture was infused into the first quadrupole of the MS/MS. Scanning in the positive ion ESI-MS revealed ions at m/z of 517 and 520 corresponding to [MH]+ of DAABD-AE derivatives of MCA and d3-MCA, respectively. Another set of more intense ions was observed at m/z of 499 and 502. These were attributed to intramolecular condensation of MCA and d3-MCA and loss of water. The transmission of these ions into the collision cell and subsequent scanning by the second resolving quadrupole for fragments revealed a simple fragmentation pattern with an intense fragment at m/z of 151 common to all studied ions. This was assigned to the N,N-dimethylaminoethylaminosulfonyl moiety originating from DAABD-AE.

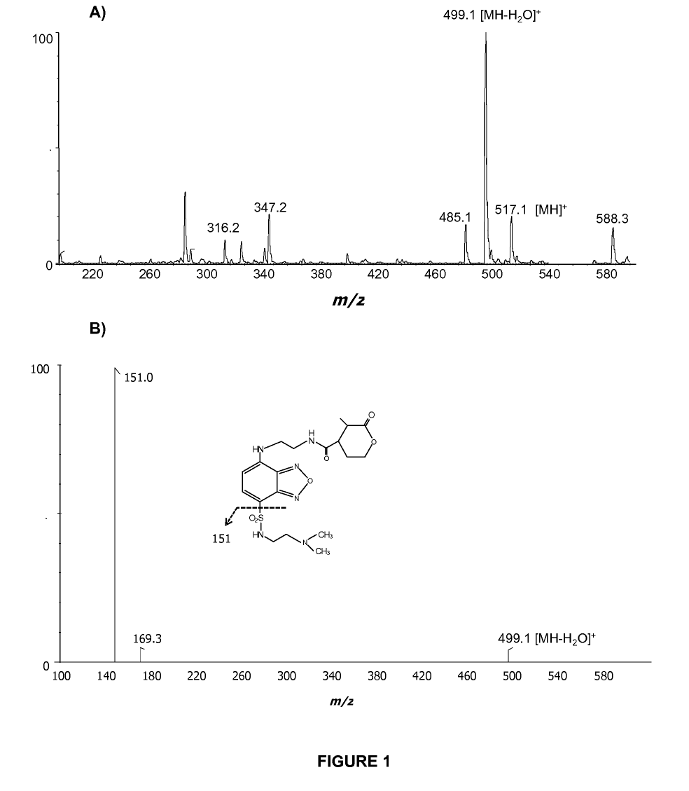

FIG. 1 shows the MS (FIG. 1A) and MS/MS (FIG. 1B) spectra of DAABD-MCA. It is noteworthy to mention that water loss may vary between different ion source designs, therefore, the use of appropriate stable isotope labeled IS is essential to compensate for this potential cause of variation.

Various chromatographic columns were evaluated aiming at the retention of DAABD-AE derivatives while allowing other compounds which may cause ion suppression including excess reagents to elute. This was achieved using a reversed phase C8 column in combination with a gradient program that increases methanol concentration from 2% to 50% (v/v) over the course of the run. The use of PFOA as a mobile phase additive improved the ionization process and enhanced the peak shape.

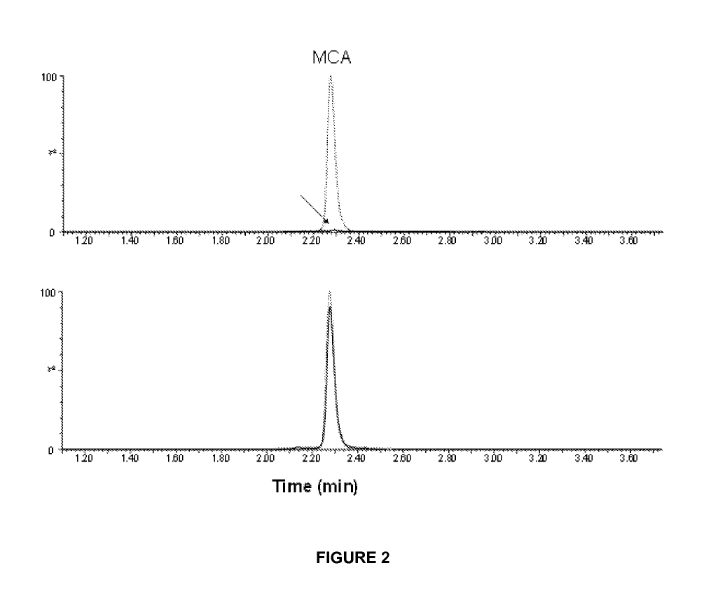

FIG. 2 shows extracted mass chromatograms obtained with a 3.2 mm DBS disc from a healthy control (FIG. 2A) and that from a patient with a MMA (FIG. 2B). As shown, MCA was well separated and eluted at 2.3 min. After each injection, the column was reconditioned for 4.3 minutes to remove ion suppression effects of late eluting compounds therefore the analytical time between successive injections was 7 min. It is worth mentioning that the automatic switching valve was programmed to divert column effluent to waste for the first 1.8 min and the last 4 min of each run to avoid loading the mass spectrometer with contaminating material from samples or reagents.

### Example 4

### Assay Validation

Linearity was established by using DBS enriched with MCA at 0.5, 1, 2, 4, 8, and 16 μmol/L. Non-enriched DBS was also analyzed to correct for endogenous MCA. Regression analysis over the studied range revealed a linear relationship (y=0.041x −0.002, r=0.9999), with y as the peak area ratio (MCA/IS) and x as the concentrations in DBS (μmol/L). The limit of quantification defined as MCA concentration that gives a signal to noise ratio (S/N) of ≧10 was found to be 0.1 μmol/L whereas the limit of detection (S/N=3) was calculated to be 0.03 μmol/L.

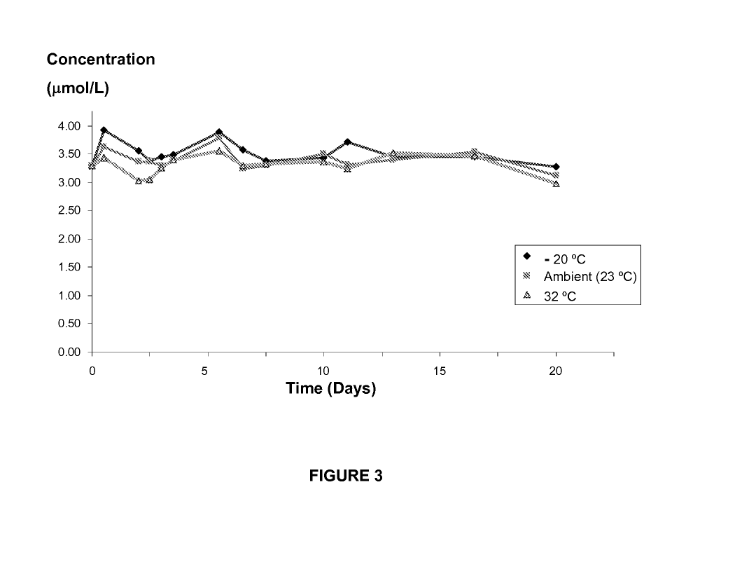

FIG. 3 depicts results of analysis of DBS specimens containing MCA at 1.5 and 3.5 μmol/L stored for a period of 3 weeks at −20° C., 23° C. (ambient) and 32° C. revealed that this compound is reasonably stable at the conditions described.

Within-day (n=19) and between-day (n=14) imprecision were evaluated by repeated analysis of QC samples at 2.5 and 10 μmol/L.

Table 1 summarizes the imprecision expressed as coefficient of variation (%) and analytical recovery.

TABLE 1
Recovery, intra- and inter-day reproducibility of MCA analysis

| | Intra-day (n = 19) | | | | Inter-day (n = 14) | | | |
|---|---|---|---|---|---|---|---|---|
| CONCENTRATION | Mean | | CV^{b} | Recovery^{c} | Mean | | CV | Recovery |
| added (μmol/L) | (μmol/L) | SD^{A} | (%) | (%) | (μmol/L) | SD | (%) | (%) |
| 2.5 | 2.2 | 0.1 | 4.5 | 88.0 | 2.3 | 0.2 | 8.7 | 92.0 |
| 10.0 | 11.4 | 0.7 | 6.1 | 114.0 | 11.5 | 0.7 | 6.1 | 115.0 |

^{A}SD = standard deviation
^{b}CV = coefficient of variation
^{c}Recovery (%) = 100 × found concentration/added concentration

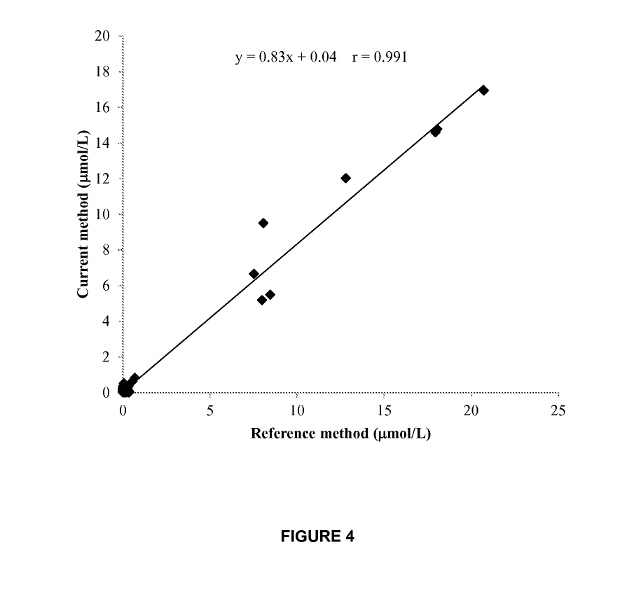

FIG. 4 illustrates the correlation between concentrations of MCA in DBS samples (n=252) measured by the current method and in parallel by a published method (Turgeon et al. 2010).

### Example 5

### Analysis of Controls and Patients Samples

Table 2 summarizes MCA concentrations obtained by the current method using DBS samples from healthy individuals (n=337) and from patients (n=20) with confirmed PA, MMA, Cbl C, Cbl F and maternal vitamin B12 deficiency. Table 2 also shows the corresponding C3 and C3/C2 levels obtained by the primary newborn screening method.

TABLE 2
Median and range of MCA, C3 and C3/C2 levels in healthy individuals
and confirmed patients studied in this work.

| | MCA (μmol/L) | C3 (μmol/L) | C3/C2 |
|---|---|---|---|
| Sample type | Median (Range) | Median (Range) | Median (Range) |
| Control (n = 337) | 0.06 (0-0.63) | 1.87 (0.46-7.15) | 0.08 (0.03-0.21) |
| Propionic acidemia (n = 2) | 10.4 (7.3-13.4) | 20.8 (16.5-25.1) | 2.3 (1.97-2.55) |
| Methylmalonyl CoA | 13.3 (5.2-19.4) | 17.6 (8.1-49.0) | 0.79 (0.38-2.06) |
| mutase deficiency (n = 8) | | | |
| Cbl C deficiency (n = 1) | 6.7 | 18.5 | 0.77 |
| Cbl F deficiency (n = 1) | 0.83 | 5.73 | 0.27 |
| Maternal vitamin B12 | 0.28 (0-2.76) | 8.4 (6.4-14.7) | 0.26 (0.14-0.40) |
| deficiency (n = 8) | | | |

### Example 6

### Discussion

As proposed by the American College of Medical Genetics, PA, MMA, Cbl A and Cbl B defects are included in the newborn screening core panel whereas Cbl C and Cbl D are considered secondary targets (Watson et al. 2006). These disorders are screened for by MS/MS using C3 as a primary marker together with appropriate ratios. Due to the overlap in C3 concentration between affected and unaffected newborns these disorders present a significant challenge to newborn screening laboratories. In affected patients, a gradient of C3 concentration is observed including subtle elevations that may be overlooked. To maximize screening sensitivity, laboratories tend to apply conservative cutoffs but this decreases test specificity resulting in a high rate of false screen positives, and potentially reveals incidental findings of non-targeted disorders. To mitigate this, second tier tests were devised to improve the screening process for this group of disorders aiming at measuring MMA, 3-hydroxypropionic acid and MCA in DBS samples (Turgeon et al. 2010; Matern et al. 2007; La Marca et al. 2007). Among these, MCA, which is traditionally detected by gas chromatography mass spectrometry as part of the organic acid profile (Rinaldo 2008) is known to accumulate in patients with defects in propionate metabolism. The MCA method is appealing because it is also capable of measuring MMA and homocysteine simultaneously. However, the MCA method of Turgeon et al (2010) as described, resulted in inadequate sensitivity and large fluctuations in reproducibility. The butylation reaction time was extended beyond 15 minutes and the formation of the tributyl MCA derivative was monitored. A 10 fold increase in peak intensity was observed at 4 hours of incubation or more. Under the modified conditions, the reproducibility was also improved, but this modification together with a lengthy chromatographic run of 15.6 min did not meet our screening objective which necessitates that samples not be batched and that reflexive testing is completed within the same working day as the initial screen positive result.

It is an aim to provide a more efficient method for MCA measurement in DBS with sufficient sensitivity, still allowing for early referral of screen positive results. MCA, a tricarboxylic acid is a hydrophilic compound with disappointing chromatographic and mass spectrometric behavior when analyzed by LC-MS/MS. To overcome this, chemical derivatization with DAABD-AE was used to generate a highly ionizable hydrophobic derivative. DAABD-AE forms stable amides upon reaction with carboxylic acids and introduces a chargeable moiety suitable for detection by ESI-MS/MS in the positive ion mode (Al-Dirbashi et al. 2007; Al-Dirbashi et al. 2008). Using reversed-phase chromatography, the hydrophobic DAABD-MCA derivative can be well separated from the early-eluting ion-suppressing compounds. The high organic content coinciding with DAABD-MCA peak elution enhanced the ionization process and contributed to the increased overall sensitivity. DAABD-AE offers outstanding reactivity and allows for derivatization using DBS specimens without the need for a dedicated extraction step. The simple sample preparation which consisted of a single 45 min step for both extraction and derivatization resulted in excellent recovery and reproducibility. With a 7 min chromatographic time, the required turn around time was met and this second tier method could be easily integrated into routine screening process. Compared with the published MCA method (Turgeon et al. 2010), our sample preparation does not require extraction and subsequent evaporation and our injection to injection time is more than halved conserving more than 50% of the instrument time.

MCA is present in DBS from healthy individuals at low, yet detectable levels (<0.7 μmol/L) (Turgeon et al. 2010). This marker is detected at significantly higher concentrations in PA or MMA patients. To obtain the maximum diagnostic value, calibrators were designed to cover a wide concentration range encompassing physiological and pathological MCA levels (0.5-16 μmol/L). The use of stable isotope IS to generate the calibration curve enhanced the quality of quantitative data obtained.

MCA levels in DBS achieved in this work were compared to those obtained by a modified version of a published LC-MS/MS method (Turgeon et al. 2010). The two methods performed adequately and showed satisfactory agreement as demonstrated by linear regression analysis shown in FIG. 4.

The potential usefulness of the proposed method as a second tier test was assessed by a retrospective study using archived DBS samples from known patients (n=12) with MMA, PA, Cbl C and Cbl F defects randomized with DBS samples from babies of maternal vitamin B12 deficiency (n=8) and healthy newborns (n=337). Median MCA concentration was 0.07 μmol/L (range 0-0.63 μmol/L) in healthy newborns. As shown in Table 2, elevated MCA was detected in all known patients regardless of the underlying genetic defect. In DBS samples from babies born to mothers with confirmed vitamin B12 deficiency (n=8), MCA was elevated in only two out of the eight samples in contrast to C3 which was elevated in all samples. With the implementation of MCA analysis as second tier test, it is expected that the false positive rate can be reduced, while maintaining excellent sensitivity. As previously reported (Turgeon et al. 2010), some patients with certain Cbl metabolic defects may not be detected by MCA analysis, hence, complementary analysis of other relevant markers such as MMA may be considered.

Many enzymes and small molecule markers are known to be stable in blood collected on filter paper as the dry nature of this matrix provides a favorable environment that decreases degradation. In this work, it was found that MCA in DBS is invariably stable for at least 3 weeks at temperatures ranging between −20° C. and 32° C. as shown in FIG. 3. This finding is significant as stability during transport of DBS samples is essential to guarantee sample integrity and result validity.

Described herein is a validated, novel, simple, and robust method to determine MCA in DBS using a single 3.2 mm disc. The excellent reactivity of the commercially available DAABD-AE reagent allowed for derivatization within 45 min, completely eliminating the extraction step. Injection to injection time was 7 min. The short sample preparation and chromatography time permits integration of this lean assay as a second tier method into routine newborn screening work flow without prolonging the turn around time. Reference intervals obtained are in agreement with the literature, and the method was able to detect confirmed cases of MMA, PA, Cbl C and Cbl F defects with 100% sensitivity. Prospective application of this method as a second tier test to improve screening for C3 related disorders is in progress.

### Example 7

### Validation Study

The forgoing method was applied in the context of a newborn screening laboratory. Between July 2011 and December 2012, a total of 222,420 DBS specimens were received. By applying a disorder algorithm based on C3 and C3/2 ratio, 103 specimens screened positive for PA or MMA and were referred for further evaluation. This revealed PA (n=3), Cbl A (n=1), Cbl C (n=5), transcobalamin II deficiency (n=1), and unclassified Cbl defect (n=1). Maternal vitamin B12 deficiency was a frequent finding (n=20) and a C3 related disorder couldn't be confirmed in the rest of these patients (n=72). In the context of the validation study, CbIC was a secondary target, while both PA and CbIA belonged to a primary screening panel. In the validation study context, transcobalamin II and unclassified Cbl defects were considered to be “incidental findings”, and were not primary or secondary targets. Accordingly, the positive predictive value (PPV) of primary and secondary screening targets using C3 and C3/C2 as screening markers was 9/103 or 8.7%.

MCA was retrospectively measured in the 103 DBS specimens that screened positive for PA or MMA, in general accordance with the above-described methods, in an attempt to reduce the false positives and incidental findings. Among these, 14 samples exceeded the set MCA cutoff of 1.0 μmol/L. These included all primary and secondary targets (n=9), unclassified Cbl (n=1), maternal vitamin B12 deficiency (n=2) and unaffected babies (n=2). The PPV obtained was therefore 64% (i.e., 9/14 having a primary or secondary target disorder) with 100% sensitivity.

Incorporating MCA testing with this cutoff threshold therefore would have eliminated 89 (about 94%) unnecessary referrals. Incorporation of MCA testing also leads to a significant reduction in the number of samples that screen positive due to maternal vitamin B12 deficiency, which may be desirable.

Lowering the MCA cutoff to 0.8 μmol/L would also have identified the transcobalamin II deficiency sample, though 6 additional patient recalls (including 5 unaffected and the one transcobalamin II deficiency sample itself) would also have occurred, thereby dropping the PPV to about 45% (this statistic includes detection of transcobalamin II deficiency as a desirable outcome). Depending on whether or not this disorder is a screening target, this reduction in PPV may or may not be justified.

Addition of the above-described method of measuring MCA to newborn screening regimes has great potential to alleviate downstream burden on a healthcare system.

### REFERENCES

Al-Dirbashi O Y, Santa T, Al-Qahtani K, Al-Amoudi M, Rashed M S (2007) Analysis of organic acid markers relevant to inherited metabolic diseases by ultra-performance liquid chromatography/tandem mass spectrometry as benzofurazan derivatives. Rapid Commun Mass Spectrom 21:1984-1990.
Al-Dirbashi O Y, Santa T, Rashed M S et al (2008) Rapid UPLC-MS/MS method for routine analysis of plasma pristanic, phytanic, and very long chain fatty acid markers of peroxisomal disorders. J Lipid Res 49:1855-1862.
Chace D H, Kalas T A, Naylor E W (2003) Use of Tandem Mass Spectrometry for Multianalyte Screening of Dried Blood Specimens from Newborns. Clinical Chemistry 49:1797-1817.
Fenton W A, Gravel R A, Rosenblatt D S (2001) Disorders of propionate and methylmalonate metabolism. In: Scriver D, Beaudet A, Valle D, Sly W (eds) The metabolic bases of inherited disease, 8th edn. McGraw-Hill Health Professions Division, New York pp 2165-93.
Fowler B, Leonard J V, Baumgartner M R (2008) Causes of and diagnostic approach to methylmalonic acidurias. J Inherit Metab Dis 31:350-60.
Gurian E A, Kinnamon D D, Henry J J, Waisbren S E (2006) Expanded newborn screening for biochemical disorders: the effect of a false-positive result. Pediatrics. 117:1915-21.
Hon D, Hasegawa Y, Kimura M, Yang Y, Verma I C, Yamaguchi S (2005) Clinical onset and prognosis of Asian children with organic acidemias, as detected by analysis of urinary organic acids using GC/MS, instead of mass screening. Brain Dev 27:39-45.
Janzen N, Peter M, Sander S et al (2007) Newborn screening for congenital adrenal hyperplasia: additional steroid profile using liquid chromatography-tandem mass spectrometry. J Clin Endocrinol Metab 92:2581-9.
La Marca G, Malvagia S, Paquini E, Innocenti M, Donati M A, Zammarchi E (2007) Rapid 2nd-tier test for measurement of 3-OH-propionic and methylmalonic acids on dried blood spots: reducing the false-positive rate for propionylcarnitine during expanded newborn screening by liquid chromatography tandem mass spectrometry. Clin Chem 53: 1364-1369.
Lindner M, Ho S, Kölker S, Abdoh G, Hoffmann G F, Burgard P (2008) Newborn screening for methylmalonic acidurias optimization by statistical parameter combination. Inherit Metab Dis 31:379-85.
Matern D, Tortorelli S, Oglesbee D, Gavrilov D, Rinaldo P (2007) Reduction of the false-positive rate in newborn screening by implementation of MS/MS-based second-tier tests: The Mayo Clinic experience (2004-2007). J Inherit Metab Dis 30: 585-592.
Minutti C Z, Lacey J M, Magera M J et al (2004) Steroid profiling by tandem mass spectrometry improves the positive predictive value of newborn screening for congenital adrenal hyperplasia. J Clin Endocrinol Metab 89:3687-93.
Rashed M S, Bucknall M P, Little D et al (1997) Screening blood spots for inborn errors of metabolism by electrospray tandem mass spectrometry with a microplate batch process and a computer algorithm for automated flagging of abnormal profiles. Clin Chem 43:1129-41.
Rinaldo P (2008) Organic Acids. In: Blau N, Duran M, Michael Gibson K (eds) Laboratory Guide to the Methods in Biochemical genetics. Springer, Heidelberg, pp 137-69.
Schulze A, Matern D, Hoffmann G F (2009) Newborn Screening. In: Sarafoglou K, Hoffmann G F, Roth K S (eds) Pediatric endocrinology and inborn errors of metabolism. McGraw-Hill Medical, New York, pp 17-32.
Tsukamoto Y, Santa T, Saimaru H, Imai K and Funatsu T (2005) Synthesis of benzofurazan derivatization reagents for carboxylic acids and its application to analysis of fatty acids in rat plasma by high-performance liquid chromatography-electrospray ionization mass spectrometry. Biomed. Chromatogr 19:802-808.
Turgeon C T, Magera M J, Cuthbert C D et al (2010) Determination of total homocysteine, methylmalonic acid, and 2-methylcitric acid in dried blood spots by tandem mass spectrometry. Clin Chem 56:1686-95.
Watkins D, Rosenblatt D S (2011) Inborn errors of cobalamin absorption and metabolism. Am J Med Genet C Semin Med Genet 157:33-44.
Watson M S, Mann M Y, Lloyd-Puryear M A, Rinaldo P, Howell R R (2006) Newborn screening: toward a uniform screening panel and system. Genet Med 8:1S-11S.
Wilcken B, Wiley V, Hammond J, Carpenter K (2003) Screening newborns for inborn errors of metabolism by tandem mass spectrometry N Engl J Med 348:2304-12.
Zytkovicz T H, Fitzgerald E F, Marsden D et al (2001) Tandem mass spectrometric analysis for amino, organic, and fatty acid disorders in newborn dried blood spots: A two-year summary from the New England newborn screening program. Clin Chem 47:1945-55.

Each reference cited herein is incorporated by reference in its entirety.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A method of measuring a level of 2-methylcitric acid (MCA) in a sample, comprising:
derivatizing the MCA with 4-[2-(N,N-dimethylamino)ethylaminosulfonyl]-7-(2-aminoethylamino)-2,1,3-benzoxadiazole (DAABD-AE);
measuring a level of the derivatized MCA; and
determining the level of MCA from the measured level of derivatized MCA.

2. The method of claim 1, wherein the sample is from a dried blood spot (DBS).

3. The method of claim 2, wherein the sample is a punch from a dried blood spot.

4. The method of claim 3, wherein the punch has a size of less than 18 mm².

5. The method of claim 3, wherein the punch has a size of less than 10 mm².

6. The method of claim 3, wherein the punch has a size of about 7 mm².

7. The method of claim 1, wherein the step of measuring is carried out by mass spectrometry.

8. The method of claim 7, wherein the mass spectrometry is liquid chromatography tandem mass spectrometry (LC-MS/MS).

9. The method of claim 1, wherein the MCA is derivatized with the DAABD-AE for less than an hour.

10. The method of claim 9, wherein the MCA is derivatized with the DAABD-AE for about 45 minutes.

11. The method claim 1, wherein the method comprises no separate extraction step prior to the step of measuring.

12. The method of claim 1, wherein the method comprises no purification step prior to the step of measuring.

13. A method of screening for a subject having an increased risk of a propionylcarnitine (C3) related disorder comprising:
derivatizing 2-methylcitric acid (MCA) of sample obtained from a subject with 4-[2-(N,N-dimethylamino)ethylaminosulfonyl]-7-(2-aminoethylamino)-2,1,3-benzoxadiazole (DAABD-AE);
measuring a level of the derivatized MCA;
comparing the level to a threshold, and
determining that the subject has an increased risk of having a propionylcarnitine (C3) related disorder in response to a determination that the level exceeds the threshold.

14. The method of claim 13, wherein the method is for screening for a subject having a C3 related disorder and the step of determining comprises determining that the subject has a C3 related disorder in response to a determination that the level exceeds the threshold.

15. The method of claim 13, wherein the sample is from a dried blood spot (DBS).

16. The method of claim 15, wherein the punch has a size of less than 10 mm².

17. The method of claim 13, wherein the step of measuring is carried out by mass spectrometry.

18. The method of claim 13, wherein the MCA is derivatized with the DAABD-AE for less than an hour.

19. The method of claim 13, wherein the method comprises no separate extraction step prior to the step of measuring.

20. The method of claim 13, wherein the method comprises no purification step prior to the step of measuring.

21. The method of claim 13, wherein the method further comprises measuring propionylcarnitine (C3) and measuring acetylcarnitine (C2).

22. The method of claim 13, wherein the C3 related disorder is propionic acidemia, a defect of cobolamin metabolism, or methylmalonic aciduria.
